(19) United States
(12) Patent Application Publication
(10) Pub. No.: US 2021/0177922 A1
(43) Pub. Date: Jun. 17, 2021

(54) PROCESS FOR PRODUCING A CANNABIS TINCTURE OR SALVE

(71) Applicant: Darrell W. Fay, Creston (CA)

(72) Inventor: Darrell W. Fay, Creston (CA)

(21) Appl. No.: 16/716,459

(22) Filed: Dec. 16, 2019

(51) Int. Cl. A61K 36/185; A61K 9/06

(52) U.S. Cl. CPC A61K 36/185; A61K 9/06; A61K 2236/15; A61K 2236/17; A61K 2236/11

(57) ABSTRACT

Processes for producing a cannabis tincture and salve are disclosed herein. The processes include steps for cultivating the required ingredients, preparing the ingredients and combining the ingredients under predetermined conditions to form a tincture, a salve, or both. The tincture and salve are useful for providing relief from a variety of ailments and discomforts.

### FIELD OF THE INVENTION

This invention relates to medicinal extracts. More particularly, it relates to cannabis-related products and extracts.

### BACKGROUND

Cannabis products have been consumed in various forms for thousands of years. The first descriptions of the medical uses date from Chinese herbal texts in the first century A.D. Cannabis products were taken orally in an herbal tea concoction and were used for their pain-relieving and sleep-inducing properties.

It was not until the middle of the nineteenth century that cannabis-based medicines were introduced into the West. The tinctures were used orally for the treatment of seizures, neuralgia, insomnia, and dysmenorrhea, among other illnesses. The cannabis-based medications were administered through an alcohol-based extract of hemp plants that were lacking in the most pharmacologically active ingredients, especially tetrahydrocannabinol (THC).

During most of the twentieth century there has been little interest in or advance of the medical use of cannabis. It has been legally prohibited in the United States since 1937. Occasionally small amounts of oil extract have been made available to some licensed university researchers for animal studies. One of the prior authors (Stephen Rosenblatt, M. D., Ph.D.) did animal research from 1969 through 1971 on learning and memory in rats, using injectable THC oil. Little to no human research on the medical uses of cannabis has been done in recent years. It is believed that, to the extent not already legalized, full state and federal legalization of cannabis related products is imminent.

In light of the foregoing, it would be desirable to devise an improved process for producing a cannabis tincture or salve that may be useful for home administration and/or treatment of discomfort relating to physical ailments.

### BRIEF DESCRIPTION OF THE DRAWINGS

FIG. 1 is a flow diagram illustrating a process of producing a cannabis tincture or salve, according to an embodiment of the present disclosure.

FIG. 2 is a flow diagram illustrating a process for producing a cannabis salve, according to an embodiment of the present disclosure.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

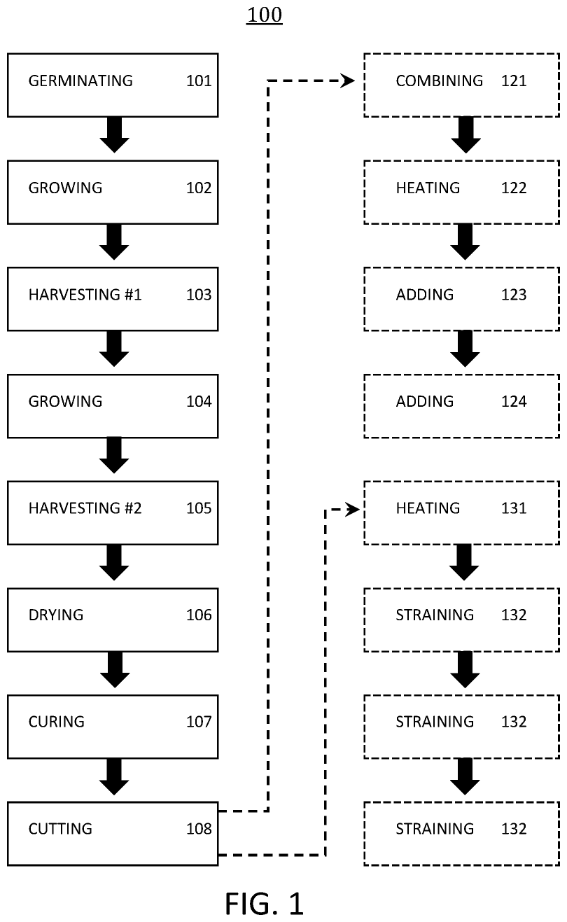

Referring to FIG. 1, a process 100 for producing a cannabis tincture or salve is disclosed. The process 100 may include the steps of germinating 101 at least one cannabis seed in organic oil. That seed may be allowed to grow 102 into at least one plant by hydrating with tea or reverse osmosis (RO) water. The seed may further be kept away from any types of pesticides or commercial fertilizers.

After a first predetermined time interval has elapsed in the growing step 102, the process may include the step of harvesting 103 leaves from a lower area of the plant. The harvested leaves secured from the harvesting step 103 may then be grown again in a supplementary growing stage 104. The resulting product may then be harvested again in a secondary harvesting step 105. The harvested leaves secured from the harvesting step 105 may then be dried 106 in a specified area for a second predetermined time interval. In a preferred embodiment, the first predetermined time interval may be 45 days, while the second predetermined time interval may be approximately 10 to 12 days. The specified area should be cool in temperature, darkened, and ventilated.

After the drying step 106 has been completed, the resulting products may go through a curing step 107. The curing step 107 may take place in a sealed container, which is then placed in a darkened area for a third predetermined time interval. The third predetermined time interval is approximately 12 to 14 days.

Finally, the product may go through a cutting stage 108, where the harvested leaves are cut into pieces. A user may then decarboxylate a first predetermined weight of said pieces at a time to create decarboxylated pieces. In a preferred embodiment, the first predetermined weight is approximately 14 grams of said pieces, while the decarboxylation of said pieces is performed in a decarboxylation unit for approximately 2 hours at approximately 220 degrees Fahrenheit.

The process 100 may additionally include the step of combining 121 a second predetermined weight (410 grams in a preferred embodiment) of organic virgin coconut oil with a third predetermined weight (64 grams in a preferred embodiment) of organic almond oil to produce an oil mixture. The oil mixture may then go through a heating step 122, wherein the oil mixture may be heated at a first predetermined temperature (approximately 145 degrees Fahrenheit) until melted. Further still, the process 100 may call for the step of adding 123 a fourth predetermined weight (approximately 42 grams) of said decarboxylated pieces to the heated oil mixture and mix them until uniform. Finally, an additional step of adding 124 a fifth predetermined weight (approximately 32 grams) of shredded bee's wax to form a salve may be called for.

In a preferred embodiment, the second predetermined weight is approximately 410 grams; the third predetermined weight is approximately 64 grams; the fourth predetermined weight is approximately 42 grams, and the fifth predetermined weight is approximately 32 grams. The process 100 may be scaled up or scaled down in weight, maintaining an approximately identical ratio between the first, second, third and fourth predetermined weights.

The process 100 may additionally include the further step of heating 131 a fifth predetermined weight (approximately 28 grams) of said decarboxylated pieces with a first predetermined liquid quantity (approximately 450 ml) of organic vegetable glycerin or MCT oil for a fourth predetermined time interval (approximately 6 hours) at a second predetermined temperature (approximately 145 degrees Fahrenheit) to form a mixture. The mixture may then go through a straining step 132 in order to separate solid parts from liquid parts. From there, the solid parts may then go through a pressing step 133, whereby remaining fluids are extracted from said solid parts. Now, the remaining fluids may be joined together with the liquid parts in a combining step 134 in order to form a tincture.

The desired amounts of included ingredients and materials described may vary in the process 100 as described above. In a preferred embodiment of the process 100, the fifth predetermined weight is approximately 28 grams and the first predetermined liquid quantity is approximately 450 ml. The process 100 may be scaled up or scaled down in weight, while maintaining an approximately identical ratio between the fifth predetermined weights and the first predetermined liquid quantity.

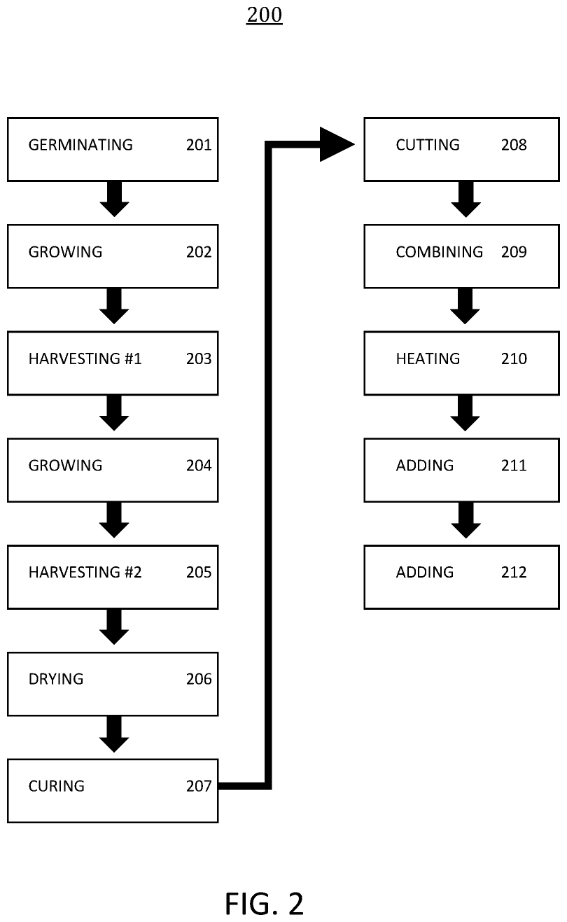

Referring to FIG. 2, a process 200 for producing a cannabis salve is disclosed. The process 200 may include the steps of: germinating 201 at least one cannabis seed in organic soil; growing 202 at least one plant by hydrating with tea or reverse osmosis (RO) water having no pesticides or fertilizers; (after approximately 45 days), harvesting 203 leaves from a lower area of the plant; growing the resulting product again in a secondary growing stage 204; harvesting 205 the growth from the secondary growing stage 204; drying 206 the harvested leaves in a cool, darkened, ventilated area for approximately 10-12 days; curing 207 the harvested leaves in a sealed container in a darkened area for approximately 12-14 days; cutting 208 the harvested leaves into pieces and decarboxylate approximately 14 grams of said pieces at a time; combining 209 approximately 410 grams of organic virgin coconut oil with a third predetermined weight of organic almond oil to produce an oil mixture; heating 210 the oil mixture at approximately 145 degrees Fahrenheit until melted; adding 211 approximately 42 grams of said decarboxylated pieces to the heated oil mixture and mix until uniform; and, adding 212 approximately 32 grams of shredded bee's wax and mix to form a salve. The items included in the combining step 209, the adding step 211 and the adding step 212 of process 200 may be scaled up or down in weight.

It should be noted that steps 121-124, as well as steps 131-134 are optional steps and may not be implemented in all cases. Optional steps of method of use or process description are illustrated using dotted/dashed lines in FIG. 1 and FIG. 2 so as to distinguish them from the other steps of methods and/or processes described. It should also be noted that some of the steps described in the processes can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other processes and/or methods, are taught herein.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I claim:

1. A process for producing a Cannabis tincture or salve comprising:
germinate at least one cannabis seed in organic soil;
grow at least one plant by hydrating with tea or reverse osmosis (RO) water, with no pesticides, or fertilizers;
after a first predetermined time interval, harvest leaves from lower area of the plant;
dry the harvested leaves in a cool, darkened, ventilated area for a second predetermined time interval;
cure the harvested leaves in a sealed container in a darkened area for a third predetermined time interval;
cut the harvested leaves into pieces and decarboxylate a first predetermined weight of said pieces at a time.

2. The process of claim 1 further comprising:
combine a second predetermined weight of organic virgin coconut oil with a third predetermined weight of organic almond oil to produce an oil mixture;
heat the oil mixture at a first predetermined temperature until melted;
add a fourth predetermined weight of said decarboxylated pieces to the heated oil mixture and mix until uniform;
add a fifth predetermined weight of shredded bee's wax and mix to form a salve.

3. The process of claim 1 wherein the first predetermined time interval is approximately 45 days.

4. The process of claim 1 wherein the second predetermined time interval is approximately 10 to 12 days.

5. The process of claim 1 wherein the third predetermined time interval is approximately 12 to 14 days.

6. The process of claim 1 wherein the first predetermined weight is approximately 14 grams.

7. The process of claim 1 wherein decarboxylation is performed in a decarboxylation unit for approximately 2 hours at approximately 220 degrees F.

8. The process of claim 2 wherein the second predetermined weight is approximately 410 grams.

9. The process of claim 2 wherein the third predetermined weight is approximately 64 grams.

10. The process of claim 2 wherein the first predetermined temperature is approximately 145 degrees F.

11. The process of claim 2 wherein the fourth predetermined weight is approximately 42 grams.

12. The process of claim 2 wherein the fifth predetermined weight is approximately 32 grams.

13. The process of claim 2 wherein the second predetermined weight is approximately 410 grams; the third predetermined weight is approximately 64 grams; the fourth predetermined weight is approximately 42 grams, and the fifth predetermined weight is approximately 32 grams.

14. The process of claim 13 wherein said process is scaled up or scaled down in weight maintaining an approximately identical ratio between the first, second, third and fourth predetermined weights.

15. The process of claim 1 further comprising:
heating a fifth predetermined weight of said decarboxylated pieces with a first predetermined liquid quantity of organic vegetable glycerin or MCT (Medium Chain Triglyceride) oil for a fourth predetermined time interval at a second predetermined temperature to form a mixture;
strain the mixture to separate solid parts from liquid parts;
press the solid parts to extract remaining fluids;
combine the remaining fluids with the liquid parts to form a tincture.

16. The process of claim 15 wherein the fifth predetermined weight is approximately 28 grams.

17. The process of claim 15 wherein the first predetermined liquid quantity is approximately 450 ml.

18. The process of claim 15 wherein the fourth predetermined time interval is approximately 6 hours;

19. The process of claim 15 wherein the second predetermined temperature is approximately 145 degrees F.

20. The process of claim 15 wherein the fifth predetermined weight is approximately 28 grams; and the first predetermined liquid quantity is approximately 450 ml.

21. The process of claim 20 wherein said process is scaled up or scaled down in weight maintaining an approximately identical ratio between the fifth predetermined weights and the first predetermined liquid quantity.

22. A process for producing a Cannabis salve comprising:
(a) germinate at least one cannabis seed in organic soil;
(b) grow at least one plant by hydrating with tea or reverse osmosis (RO) water, with no pesticides, or fertilizers;
(c) after approximately 45 days, harvest leaves from lower area of the plant;
(d) dry the harvested leaves in a cool, darkened, ventilated area for approximately 10-12 days;
(e) cure the harvested leaves in a sealed container in a darkened area for approximately 12-14 days;
(f) cut the harvested leaves into pieces and decarboxylate approximately 14 grams of said pieces at a time.
(g) combine approximately 410 grams of organic virgin coconut oil with a third predetermined weight of organic almond oil to produce an oil mixture;
(h) heat the oil mixture at approximately 145 degrees F. until melted;
(i) add approximately 42 grams of said decarboxylated pieces to the heated oil mixture and mix until uniform;
(j) add approximately 32 grams of shredded bee's wax and mix to form a salve.

23. The process of claim 22 wherein steps (g), (i) and (j) are scaled up or down in weight.
